(19) United States
(12) Patent Application Publication
(10) Pub. No.: US 2004/0199201 A1
(43) Pub. Date: Oct. 7, 2004

(54) Embolectomy devices

(75) Inventors: James Kellett, Los Gatos, CA (US); Valerie Sontrop, Palo Alto, CA (US); Greg Welsh, Los Gatos, CA (US); Dushyant Jivanlal Shah, San Ramon, CA (US); Kamal Ramzipoor, Fremont, CA (US); Mehran Bashiri, San Carlos, CA (US); Ajitkumar Nair, Fremont, CA (US); Andrew Huffmaster, Fremont, CA (US); Thomas Yung-Hui Chien, San Jose, CA (US); Mark Minh Phung, Union City, CA (US); Mediko Issakhani, Gilroy, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SCIMED LIFE SYSTEMS, INC.

(21) Appl. No.: 10/698,760

(22) Filed: Oct. 30, 2003

Related U.S. Application Data
(60) Provisional application No. 60/460,630, filed on Apr. 2, 2003.
(60) Provisional application No. 60/460,586, filed on Apr. 2, 2003.

(51) Int. Cl. A61M 29/00

(52) U.S. Cl. 606/200; 606/191

(57) ABSTRACT

Devices and methods for removing a foreign object from a body lumen are disclosed. An embolectomy device can include a filter basket having a plurality of filter struts forming a cage-like structure that can be expanded to circumferentially surround the incoming foreign object. A support frame including a proximal hoop and one or more rail members may be employed to support the filter basket. The support frame and filter basket can be coupled to a pusher wire that can be used to manipulate the device within the body. In certain embodiments, the filter struts may vary in thickness to impart a desired flexibility characteristic to the device. Methods of manufacturing such devices are also disclosed.

### CROSS REFERENCE TO RELATED APPLICATION

The present application claims benefit to provisional U.S. Patent Application Nos. 60/460,586 and 60/460,630, both filed on Apr. 2, 2003.

### FIELD OF THE INVENTION

The present invention relates generally to the field of medical devices. More specifically, the present invention pertains to embolectomy devices for removing foreign objects within a body lumen.

### BACKGROUND OF THE INVENTION

Embolectomy devices such as inflatable catheters and clot pullers are used in a variety of applications to remove blood clots or other foreign objects from a blood vessel. In applications involving the cerebrovasculature, for example, such devices may be used to remove a blood clot from an intracranial artery for the treatment of ischemic stroke. The formation of thrombus within the artery may partially block or totally occlude the flow of blood through the artery, preventing blood from reaching the brain or other vital organs. Such thrombolytic events may also be exacerbated by atherosclerosis, a vascular disease that causes the vessels to become tortuous and narrowed. The tortuosity or narrowness of the vessel may, in certain circumstances, lead to the formation of atherosclerotic plaque, which can cause further complications to the body if not treated.

In embolectomy procedures for removing blood clots, a delivery catheter or sheath is typically inserted percutaneously into the body (e.g. via the femoral, jugular or antecubital veins) and advanced to a target site within the body containing the clot. To ascertain the precise location of the clot within the body, a radiopaque die can be injected into the body to permit the occluded vessel to be radiographically visualized with the aid of a fluoroscope. A Fogarty catheter or other suitable delivery device can be used to transport the embolectomy device in a collapsed position distal the site of the blood clot. The embolectomy device is then deployed, causing the embolectomy device to expand in the vessel. The embolectomy device can then be urged in the proximal direction to remove the clot from the vessel wall, if necessary. A wire basket, coil, membrane or other collector element can be used to capture the clot as it is dislodged from the vessel wall. Once entrained within the collector element, the embolectomy device and captured blood clot are then loaded into a retrieval device and withdrawn from the patient's body.

The efficacy of the embolectomy device to dislodge the blood clot from the vessel wall depends in part on the mechanical strength of the collector element. In an embolectomy device employing basket-type filters, for example, the proximal section of the device must have sufficient strength to support the filter basket in an expanded position while the blood clot is dislodged from the vessel wall. An insufficient amount of strength at the proximal section of the device may, in certain circumstances, cause the filter basket to deflect away from the vessel wall at the site of the blood clot. As a result, the ability of the embolectomy device to dislodge and subsequently capture the clot may be compromised.

### SUMMARY OF THE INVENTION

The present invention pertains to embolectomy devices for removing foreign objects within a body lumen. An embolectomy device in accordance with an exemplary embodiment of the present invention can include a support frame having a proximal hoop and at least one rail member configured to support a flexible filter basket within the blood vessel. A portion of the support frame may be attached to an elongated member that can be manipulated during an embolectomy procedure to dislodge the foreign object from the vessel wall.

The filter basket may be actuatable between a collapsed position and an expanded position. In certain embodiments, the filter basket can be biased to self-expand when deployed in the vessel, either by a mechanical force imparted to the device, or from the use of superelastic alloys treated to exhibit certain shape-memory properties. The filter basket can include a number of filter struts of reduced dimension. A proximal set of filter struts may be employed to attach a proximal section of the filter basket to the support frame. A distal set of filter struts can be employed to attach a distal section of the filter basket and the distal end of each rail member to a bushing disposed about the elongated member.

In certain embodiments, the filter basket can include a plurality of interconnected filter struts formed from a single workpiece such as a tube, foil or sheet. The filter struts can be arranged to form a number of filter cells configured circumferentially to surround the incoming foreign object. The filter cells can also be configured to displace in multiple directions, if desired. In certain embodiments, a polymeric web covering can be placed about all or a portion of the filter basket.

The filter struts forming the filter basket can vary in flexibility to impart a particular flexibility characteristic to the embolectomy device. In some embodiments, for example, a proximal section of the filter basket can include filter struts having a relatively large cross-sectional area to impart greater mechanical strength to the portion of the embolectomy device that dislodges the foreign object from the vessel wall. The distal section of the filter basket, in turn, can include one or more struts of reduced thickness for increased flexibility as the device is advanced through the body. One or more radiopaque features may be employed to visualize the positioning and deployment status of the embolectomy device within the blood vessel.

In an exemplary method of manufacture, a workpiece of uniform thickness tubing, foil or flat sheet can be laser-cut or photo-chemically etched to form the various filter struts and support hoop of the filter basket. Selective portions of the filter basket may be masked, and a suitable reduction process such as microblasting or electropolishing may be performed to reduce the wall thickness at the unmasked areas of the filter basket. In certain embodiments, the filter struts forming the distal section of the filter basket can be reduced in thickness to impart flexibility to the distal section of the embolectomy device to aid in the advancement of the device through tortuous or narrowed vessels. Selective filter struts forming the proximal section of the filter basket can be masked to maintain their original thickness, thereby imparting greater mechanical strength to the proximal section of the embolectomy device.

### BRIEF DESCRIPTION OF THE DRAWINGS

FIG. 1 is a perspective view of an embolectomy device in accordance with an exemplary embodiment of the present invention employing a support frame and filter basket;

FIG. 2 is an end view of the support frame illustrated in FIG. 1;

FIG. 3 is a side view of the support frame illustrated in FIG. 1;

FIG. 4 is a top view of the support frame illustrated in FIG. 1;

FIG. 5 is a top view of the filter basket of FIG. 1, showing the filter basket prior to assembly on the pusher wire;

FIG. 6 is a perspective view of an embolectomy device in accordance with another exemplary embodiment of the present invention employing a support frame and filter basket;

FIG. 7 is a perspective view of an embolectomy device in accordance with an exemplary embodiment of the present invention having a unitary filter basket construction;

FIG. 8 is a top view of the filter basket of FIG. 7, showing the filter basket prior to assembly on the pusher wire;

FIG. 9 is another top view of the filter basket of FIG. 7, showing the filter basket with a polymeric web covering;

FIG. 10 is a partial cross-sectional view showing the embolectomy device of FIG. 1 collapsed within a delivery device and advanced to a target region within a blood vessel;

FIG. 11 is a partial cross-sectional view showing the embolectomy device of FIG. 1 in a second position deployed from the delivery device;

FIG. 12 is a partial cross-sectional view showing the embolectomy device of FIG. 1 in a third position engaged within the blood vessel;

FIG. 13 is a partial cross-sectional view showing the embolectomy device and captured blood clot withdrawn into the delivery device;

FIG. 14 is a perspective view of an embolectomy device in accordance with an exemplary embodiment of the present invention having a filter basket with variable flexibility;

FIG. 15 is a detailed view of a portion of the proximal section of the filter basket illustrated in FIG. 14;

FIG. 16 is a detailed view of a portion of the distal section of the filter basket illustrated in FIG. 14;

FIG. 17 is a partial cross-sectional view showing the embolectomy device of FIG. 14 collapsed within a delivery device and advanced to a target region within a blood vessel;

FIG. 18 is a partial cross-sectional view showing the embolectomy device of FIG. 14 in a second position deployed from the delivery device;

FIG. 19 is a partial cross-sectional view showing the embolectomy device of FIG. 14 in a third position engaged within the blood vessel; and

FIG. 20 is a partial cross-sectional view showing the embolectomy and captured blood clot withdrawn into the delivery device.

### DETAILED DESCRIPTION OF THE INVENTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

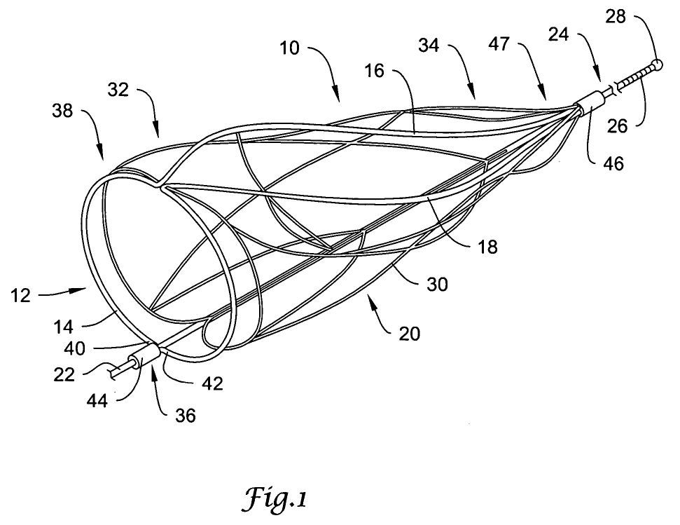

FIG. 1 is a perspective view of an embolectomy device 10 in accordance with an exemplary embodiment of the present invention. As shown in FIG. 1, embolectomy device 10 can include a support frame 12 forming a proximal hoop 14 and one or more rail members 16,18, a filter basket 20 operatively coupled to the support frame 12, and a pusher wire 22 that can be manipulated within the body to engage the embolectomy device 10.

The pusher wire 22 can include a distal section 24 configured to support the support frame 12 and filter basket 20 within a blood vessel, and a proximal section (not shown) configured to lie outside of the patient's body. The pusher wire 22 can be configured similar to other guiding members used in the art (e.g. guidewires), having the ability to transmit axial and rotational motion from the proximal section of the wire to the distal section. The pusher wire 22 may be tapered slightly such that the distal section 24 of the pusher wire 22 has a smaller profile than the proximal section. A radiopaque spring coil 26 disposed about the distal section 24 of the pusher wire 22 may provide additional stiffness to the pusher wire 22 while providing a visual reference point when used in conjunction with a fluoroscope. An atraumatic distal tip 28 having a bulbous shape may also be employed, if desired, to reduce trauma to the body.

The filter basket 20 can include a number of filter struts 30 that form a cage-like structure configured to capture the incoming foreign object. A proximal set of filter struts 32 can be used to attach the filter basket 20 to the rail members 16,18 of the wire frame 12. In addition, a distal set of struts 34 can be used to couple the filter basket 20 to the distal section 24 of the pusher wire 22.

The proximal hoop 14 can be secured to the distal section 24 of the pusher wire 22 via a joint 36 located adjacent to a proximal section 38 of the embolectomy device 10. In certain embodiments, joint 36 may be formed by soldering, brazing, welding, crimping, adhering, or otherwise bonding the ends 40,42 of the proximal hoop 14 to a tubular segment 44 secured to the pusher wire 22. In an alternative embodiment (not shown), the ends 40,42 of the proximal hoop 14 can be attached directly to the pusher wire 22.

A bushing 46 disposed about the pusher wire 22 at or near a distal section 47 of the embolectomy device 10 connects the distal set of struts 34 and rail members 16,18 to the pusher wire 22. Bushing 46 may have an inner lumen configured to slidably receive the pusher wire 22, allowing the support frame 12 and filter basket 20 to move back and forth along the pusher wire 22 as the embolectomy device 10 is actuated between the collapsed and expanded positions. The bushing 46 can be attached to the distal set of struts 34 and rail members 16,18 with an epoxy or other suitable bonding agent.

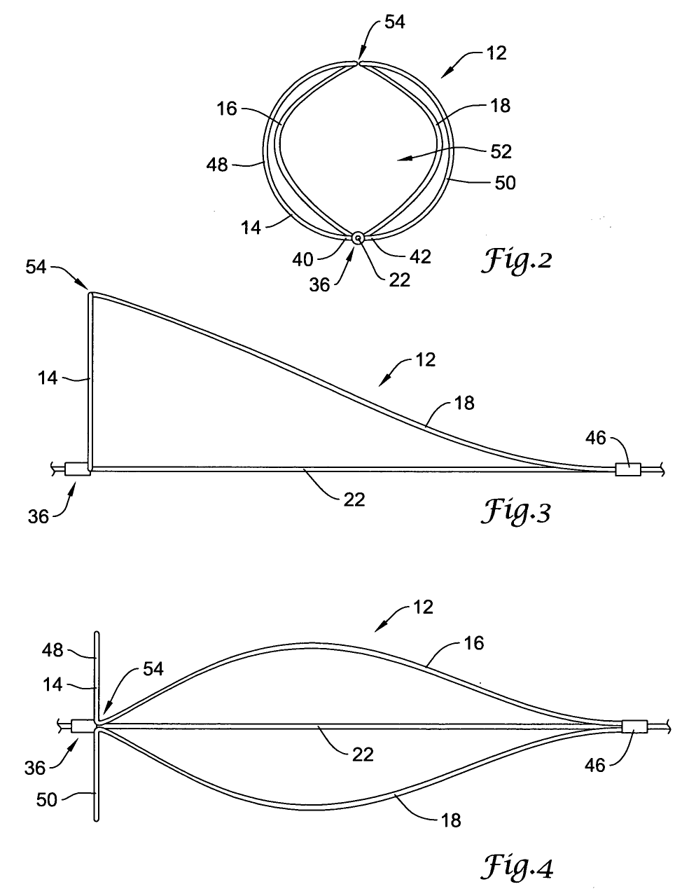

Turning now to FIGS. 2-4, the support frame 12 illustrated in FIG. 1 will now be described in greater detail. Support frame 12 is configured to support the filter basket 20 in an expanded position when deployed in the body, but has sufficient elasticity to permit the embolectomy device 10 to be radially collapsed within the lumen of the delivery device (e.g. a microcatheter or guide catheter). The support frame 12 can be configured to self-expand when deployed in the body, or can be configured to manually expand with the aid of a mandrel or other actuator mechanism. The support frame 12 can be constructed from two separate members 48,50 coupled together at each respective end 40,42 at joint 36. As can be seen in FIGS. 2 and 4, the left member 48 forming the left portion of the proximal hoop 14 has a semi-circular shape that is oriented in a plane substantially perpendicular to the longitudinal axis of the pusher wire 22. In similar but mirrored fashion, the right member 50 forming the right portion of the proximal hoop 14 also has a semi-circular shape that is oriented in a plane substantially perpendicular to the longitudinal axis of the pusher wire 22. Together, the semi-circular portions of the left and right members 48,50 define an opening or mouth 52 of the embolectomy device 10 that receives the incoming foreign object.

At location 54, the left and right members 48,50 both bend and orient in a direction towards the distal section 47 of the embolectomy device 10, forming the rail members 16,18. As can be seen in FIGS. 3-4, the rail members 16,18 may each have an arcuate shape that bows outwardly while sloping downwardly towards the bushing 46. In use, the rail members 16,18 provide added radial and longitudinal stiffness to the embolectomy device 10.

The left and right members 48,50 may each be formed of wire or ribbon having a size and shape configured to provide a desired amount of stiffness to the embolectomy device 10. In certain embodiments, for example, the left and right members 48,50 can have a circular transverse cross-sectional area having a diameter in the range of about 0.003 to 0.004 inches, although other sizes and shapes may be employed, if desired. The left and right members 48,50 can be formed from a metal, polymer, or metal-polymer blend selected to exhibit certain mechanical characteristics such as torsional rigidity and stiffness. In certain embodiments, the left and right members 48,50 can be formed from a superelastic material such as a nickel-titanium alloy (Nitinol), allowing the embolectomy device 10 to be collapsed into relatively small delivery devices such as a microcatheter or the like. The superelastic material can be treated to exhibit certain shape-memory properties when deployed in the body. For example, the members 48,50 can be heat-treated to revert from a collapsed position having a relatively small profile to an expanded position such as that depicted in FIGS. 2-4.

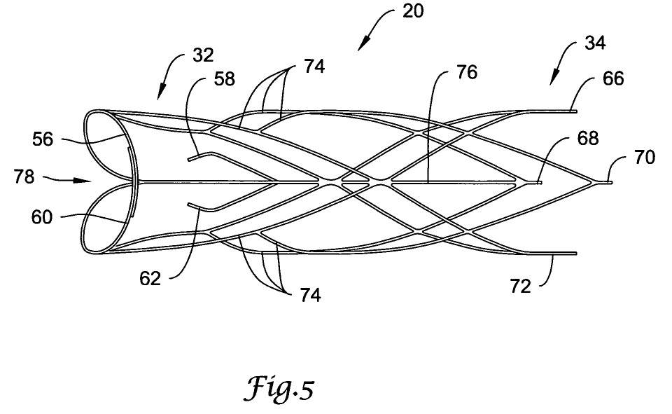

FIG. 5 is a top view of the filter basket 20 illustrated in FIG. 1 prior to being assembled on the pusher wire 22. As illustrated in FIG. 5, the proximal set of struts 32 may include four struts 56,58,60,62 which together connect the filter basket 20 to the support frame 12. During assembly, the left rail member 16 may be attached to the filter basket 20 via struts 56 and 58. Similarly, the right rail member 18 may be attached to the filter basket 20 via struts 60 and 62. In certain embodiments, the struts 56,58 used to attach the filter basket 20 to the left rail member 16 may have a degree of symmetry with the struts 60,62 used to attach the filter basket 20 to the right rail member 18. As with the support frame 12, the filter basket 20 may be biased to automatically shift from a collapsed position to an expanded position when deployed in the body.

Although the four struts 56,58,60,62 depicted in FIG. 5 are configured to attach to the rail members 16,18, other attachment locations are possible. In one alternative, for example, the struts 58,62 arising from the bottom of the filter basket 20 may be attached to various locations on the proximal hoop 14. The number of attachment points may also vary to impart more or less flexibility to the filter basket 20, as desired. Thus, while four struts 56,58,60,62 are specifically illustrated in FIG. 5, a greater or lesser number of struts can be employed to connect the filter basket 20 to the support frame 12.

The distal set of struts 34 can include four struts 66,68,70,72 which together connect the distal end of the filter basket 20 to the pusher wire 22. The four struts 66,68,70,72 can be oriented to converge in symmetrical fashion at the bushing 46, thereby closing the distal section 47 of the embolectomy device 10 to prevent the escape of the foreign object. When assembled, the filter basket 20 has a generally conical shape with its apex located adjacent to the proximal hoop 14 of the support frame 12. As with the proximal set of struts 32, the number of struts employed may vary to alter the filtering characteristics of the filter basket 20.

As can be further seen in FIG. 5, filter basket 20 may also include a number of other filter struts 74 oriented in various positions along the length of the device 10. The filter struts 74 can be interconnected via several attachment locations, forming a cage-like structure configured to capture emboli while maintaining the perfusion of blood through the vessel. A strut 76 extending along the bottom portion of the filter basket 20 adjacent to the pusher wire 22 forms a spine of the filter basket 20 that can be used in conjunction with other filter struts to support the filter basket 20. A proximal portion of strut 76 splits and bends upwardly towards the top portion of the filter basket 20, forming a hoop 78 configured to lie adjacent to the proximal hoop 14 of the support frame 12.

In certain embodiments, the thickness of the various struts used in forming the filter basket 20 can be made thinner than the thickness of the rail members 16,18 to impart greater flexibility to the filter basket 20. For example, at least one of the filter struts forming the filter basket 20 can have a diameter of about 0.002 inches whereas the members 48,50 used to form the proximal hoop 14 and rail members 16,18 can have a larger diameter of about 0.003 to 0.004 inches.

The embolectomy device 10 can include one or more radiopaque features which allow the device to be visualized within the body using a fluoroscope. For example, one or more radiopaque coils or marker bands placed on selective locations of the embolectomy device 10 may be used to identify the location of the device 10 in the body. In certain embodiments, for example, a radiopaque coil formed of platinum can be placed about the proximal hoop 14 and/or rail members 16,18 which, when viewed with a fluoroscopic monitor, allow the operator to determine the location and status (i.e. deployed or collapsed) of the embolectomy device 10.

The manufacturing of the filter basket 20 as well as other components of the embolectomy device 10 can be accomplished by a number of different methods and techniques. In certain techniques, for example, a tubular workpiece may be cut and/or etched to form the various struts of the filter basket 20. Alternatively, a foil or flat sheet of material can be cut and/or etched, and then rolled into a tubular shape and bonded along a seam or attached to a wire to form the filter basket 20. An electropolishing process or other suitable technique may be used to provide a smooth finish to the final, cut filter basket 20. In some embodiments, a hydrophilic, hydrophobic or other suitable coating can be placed on the filter basket 20 and/or other components of the embolectomy device 10 to reduce friction or other restrictive force as the device is advanced through the body or placed into contact with the delivery device.

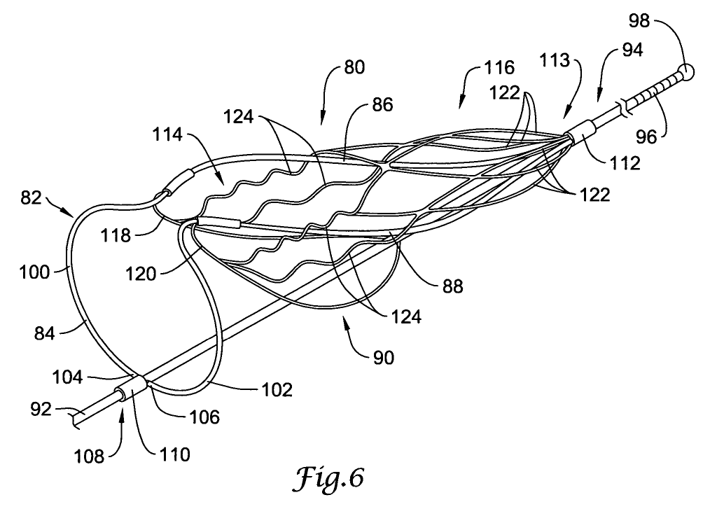

FIG. 6 is a perspective view of an embolectomy device 80 in accordance with another exemplary embodiment of the present invention. Embolectomy device 80 can include a support frame 82 forming a proximal hoop 84 and one or more rail members 86,88, a filter basket 90 operatively coupled to the support frame 82, and a pusher wire 92 that can be manipulated by the operator at a location outside of the patient's body to engage the embolectomy device 80 within the body.

The support frame 82 and pusher wire 92 can be configured similar to the support frame 12 and pusher wire 22 described above with respect to FIGS. 1-4. The distal section 94 of pusher wire can be distally tapered, and can include a radiopaque spring coil 96 and atraumatic distal tip 98. The support frame 82 can be constructed from two separate members 100,102 coupled together at their respective proximal ends 104,106 at joint 108, and can be shaped to form the proximal hoop 84 and rail members 86,88. Joint 108 can be formed by soldering, brazing, welding, crimping, adhering, or otherwise bonding the proximal ends 104,106 of the two members 100,102 to a tubular segment 110 secured to the pusher wire 92. A bushing 112 slidably disposed about the pusher wire 92 at or near a distal section 113 of the embolectomy device 80 connects the filter basket 90 and rail members 86,88 to the pusher wire 92.

The filter basket 90 can include a proximal set of struts 114 that attach the filter basket 90 to the rail members 86,88, and a distal set of struts 116 that couple the filter basket 90 to the distal section 94 of the pusher wire 92. As shown in FIG. 6, the proximal set of struts 114 can include a left strut 118 and a right strut 120. The left and right struts 118,120 may each be connected, respectively, to the left and right rail members 86,88. The left and right struts 118,120 can, however, be attached to other locations along the support frame 82, including the proximal hoop 84.

The distal set of struts 116 can include six struts 122 that converge and attach to the bushing 112 in symmetrical fashion, thus closing the distal section 113 of the embolectomy device 80. As with the proximal set of struts 114, the number and relative orientation of each of the distal set of struts 116 can vary to alter the containment characteristics of the filter basket 90, if desired.

In addition to the proximal and distal set of struts 114,116, filter basket 90 can include a number of other filtering struts 124 forming a cage-like structure configured to capture emboli while maintaining the perfusion of blood through the vessel. The filtering struts 124 can be oriented in a generally longitudinal direction along the length of the filter basket 90, and can have an undulating shape that grips the foreign object as it is captured. In certain embodiments, greater flexibility can be imparted to the filter basket 90 by reducing the thickness of the filter struts 124 as well as the proximal and distal sets of struts 114,116. Such flexibility allows the various filter struts to easily bend or flex when the incoming clot is received, allowing the device 80 to capture the foreign object without severing or breaking the object into smaller fragments.

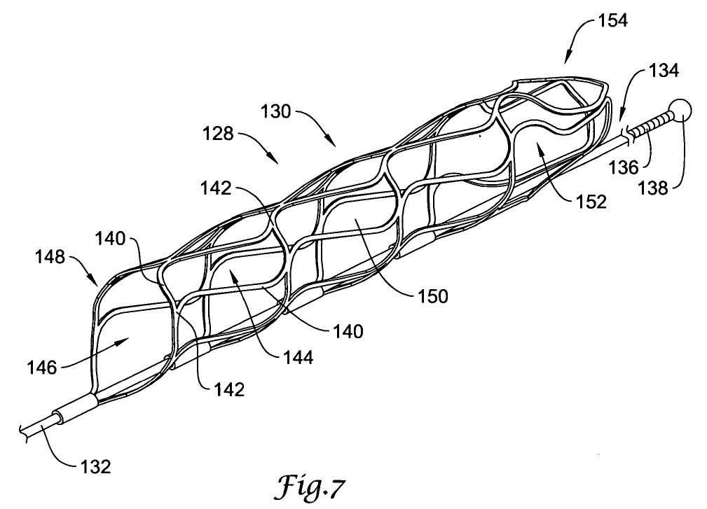

FIG. 7 is a perspective view of an embolectomy device 128 in accordance with another exemplary embodiment of the present invention having a unitary construction. As shown in FIG. 7, embolectomy device 128 can include a filter basket 130 operatively coupled to a pusher wire 132 that can be manipulated at a location outside of the patient's body to engage the embolectomy device 128. The pusher wire 132 can be configured similar to pusher wire 22 discussed herein, having a distal section 134 that is distally tapered, and including a radiopaque spring coil 136 and atraumatic distal tip 138.

The filter basket 130 can include several filter struts 140 and connecting junctures 142 that form a number of basket cells 144 configured circumferentially to surround and capture the foreign object therein. The filter basket 130 can include an opening 146 in a proximal section 148 of the embolectomy device 128, which receives the incoming foreign object as it is dislodged from the vessel wall. The basket opening 146 can be configured to grip or pinch the foreign object when the embolectomy device 128 is withdrawn slightly into the distal end of the delivery device. The basket cells 144 located on the proximal section 148 of the embolectomy device 128 can be arranged in a circumferential manner, forming an inner lumen 150 within the filter basket 130 that receives the incoming foreign object. Several basket cells 152 located at a distal section 154 of the filter basket 130 can have a closed configuration, preventing the foreign object or other emboli from escaping from the filter basket 130 once captured therein.

The basket opening 146 may have a scoop-like shape that, when engaged along the vessel wall, dislodges the clot without slipping. The size of the opening 146 can be selected to engage foreign objects at various locations within the vasculature, such as at bifurcated locations. The profile of the filter basket 130 can be generally cylindrical, conical, or other desired shape.

The filter struts 140 forming the basket cells 144 can be configured to move and expand in multiple directions. In a first direction, the filter struts 140 can be configured to act in a radial direction, providing an outward force to aid in expansion of the device 128 within the vessel. In a second direction, strut 140 compression can be reduced when an axial load is asserted along the longitudinal axis of the device 128. In a third direction, the filter struts 140 along the top portion of the device 128 located furthest away from the pusher wire 132 may be configured to move more in the longitudinal direction than the filter struts 140 located immediately adjacent to the pusher wire 132, thereby imparting a bending or folding movement to the embolectomy device 128. In use, this bending or folding movement allows the junctures 142 of the filter basket 130 to be more evenly dispersed, imparting greater flexibility, a lower profile, and reduced friction to the embolectomy device 128. As with previous embodiments, the filter struts 140 can be electro-polished and/or can include a hydrophilic or hydrophobic coating, further improving the deliverability of the device 128.

In certain embodiments, the filter struts 140 can include a superelastic material such as a nickel-titanium alloy (Nitinol) having certain shape-memory properties that permit the embolectomy device 128 to revert to a particular shape when exposed to a certain temperature within the body. In certain embodiments, for example, the filter struts 140 may be made from a superelastic material having an Aₛ-A_{f} transition temperature set above body temperature (e.g. at 40-50° C.). The material can be heat-set such that the filter basket 130 remains collapsed at temperatures below the final austenitic temperature A_{f} of the material, thus imparting less radial force on the inner wall of the delivery device during delivery. The embolectomy device 128 can be loaded into the distal end of the delivery device in its unexpanded form, and delivered to a target site within a vessel. An infusion of warm saline or other suitable fluid can then be injected into the lumen of the delivery device, transforming the filter basket 130 from a collapsed position to an expanded position within the vessel.

FIG. 8 is a top view of the filter basket 130 of FIG. 7, showing the filter basket 130 prior to assembly on the pusher wire 132. As shown in FIG. 8, the filter basket 130 can have a unitary construction formed from a single workpiece, reducing the number of components necessary to form the device. A laser machining, laser etching, chemical etching, or photochemical etching process can be used to cut the workpiece to form the various elements of the device. Once formed, a thin layer of polytetraflouroethylene (PTFE) may be placed about the filter basket 130 to reduce friction and slippage as the embolectomy device 128 is advanced within the vessel. Radiopaque markers can also be placed at selective locations on the device 128 to enhance radiographic visualization using a fluoroscope. Special inlet cuts or recesses on the filter struts 140 may be used to attach the radiopaque markers to the filter basket 130 without increasing the profile of the device.

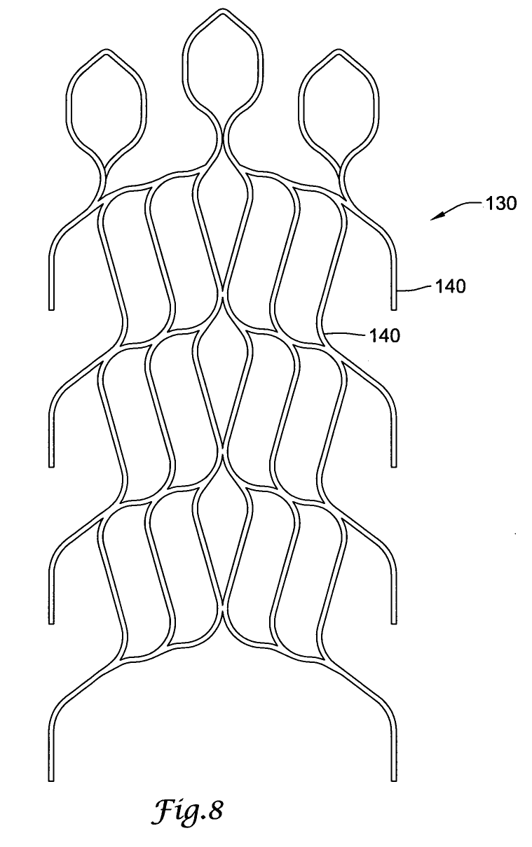

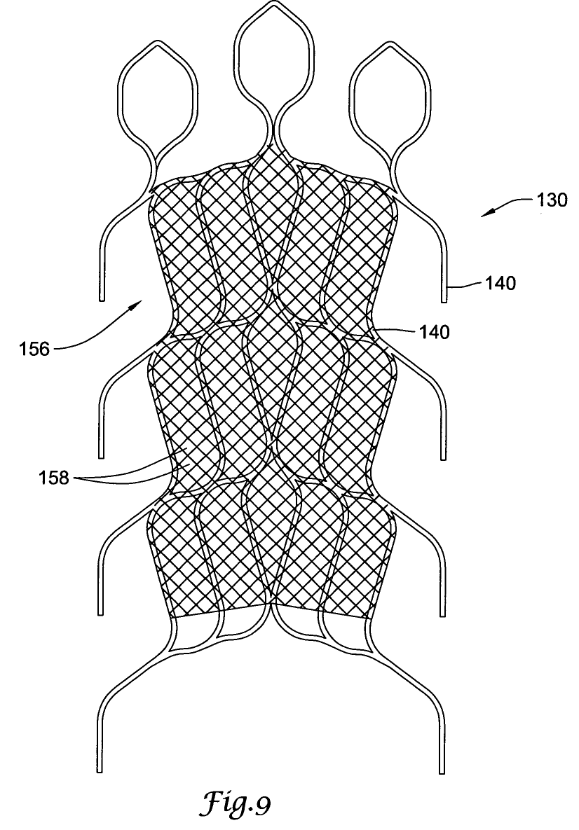

The filter basket 130 may further include a polymeric web covering to further capture the foreign object or any other emboli therein. As shown in FIG. 9, for example, a polymeric web 156 of, for example, expanded polytetraflouroethylene (PTFE) can be coupled to selective filter struts 140 on the filter basket 130. The polymeric web 156 can include a number of openings or pores 158 of sufficient size to capture the foreign object and any emboli while maintaining the perfusion of blood through the filter basket 130.

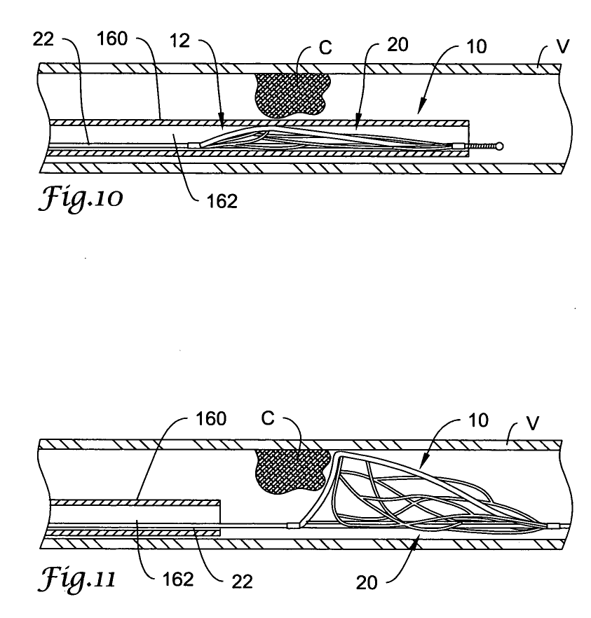

Referring now to FIGS. 10-13, an exemplary method of retrieving a foreign object within a blood vessel will now be described with respect to embolectomy device 10 described herein. Embolectomy device 10 may be loaded into a delivery device 160 having an internal lumen 162 configured to receive the device 10 in a collapsed position. The embolectomy device may be loaded into the lumen 162 of the delivery device 160 by inserting the proximal end of the pusher wire 22 into the lumen 162, and then urging the embolectomy device 10 into lumen 162 such that the support frame 12 and filter basket 20 collapse therein. Once loaded, the delivery device 160 and collapsed embolectomy device 10 can then be inserted percutaneously into the body and advanced to a target region within the vessel V distal to a blood clot C, as shown in FIG. 10.

After being positioned at the target site, the embolectomy device 10 can then be deployed from within the delivery device 10, causing the device 10 to expand within the blood vessel V, as shown in FIG. 11. The filter basket 20 may have an expanded size that approximates the size of the blood vessel V to provide full apposition therein. In those embodiments employing shape-memory alloys, a warm saline solution may be delivered through lumen 162 and placed into contact with the embolectomy device 10, causing the material to transform to austenite and recover its pre-formed (i.e. expanded) shape. Alternatively, the shape-memory material may be configured to transform to austenite at body temperature (i.e. about 37° C.), in which case the exposure of the embolectomy device 10 to blood within the blood vessel V causes the device to revert to its expanded shape.

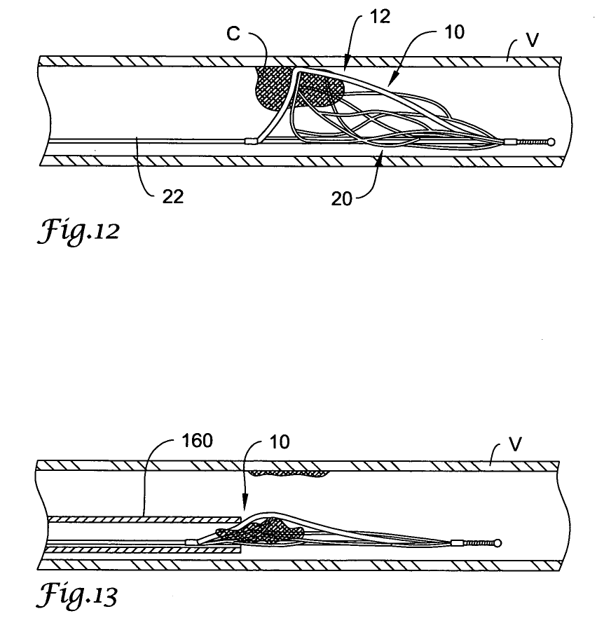

Once deployed in the blood vessel V, the embolectomy device 10 can then be pulled proximally a distance to dislodge the blood clot C from the vessel V, as shown in FIG. 12. As can be seen in FIG. 12, the support frame 12 maintains the rigidity of the embolectomy device 10 as it is urged proximally along the vessel wall. The engagement of the embolectomy device 10 shears the blood clot C from the vessel wall, forcing the blood clot C through the proximal hoop 14 and into the filter basket 20. After the blood clot C has been captured within the filter basket 20, the embolectomy device 10 is then withdrawn back into the delivery device 160, as shown in FIG. 13. The delivery device 160 and accompanying embolectomy device 10 can then be removed from the body.

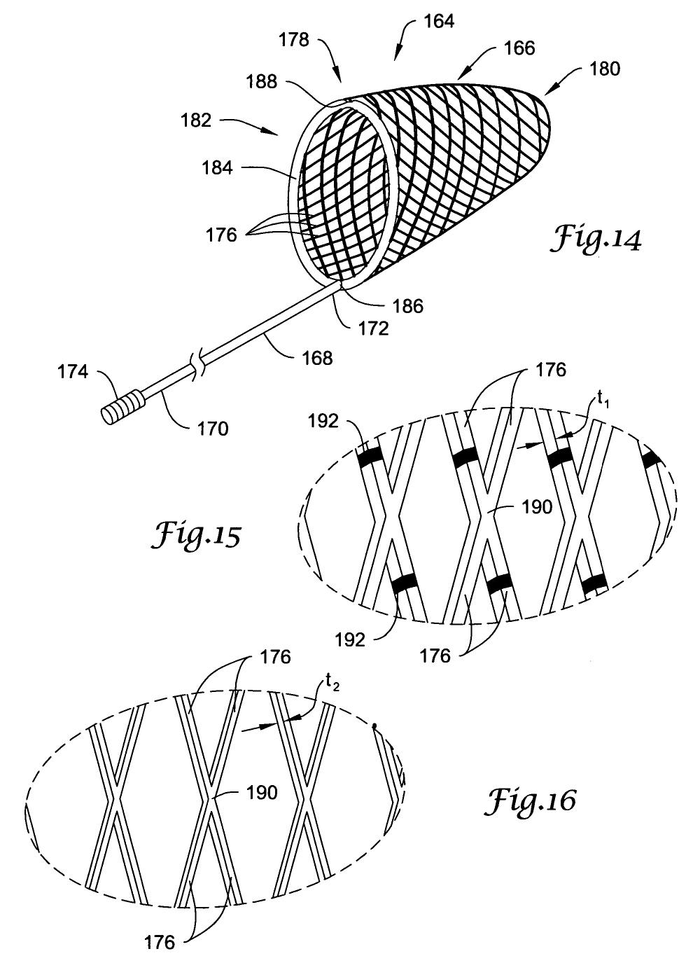

FIG. 14 is a perspective view of an embolectomy device 164 in accordance with an exemplary embodiment of the present invention employing a filter basket with variable flexibility. Embolectomy device 164 can include a filter basket 166 operatively coupled to an elongated member 168 having a proximal section 170 and a distal section 172. Elongated member 168 can include a guide wire, push rod or other like device configured to transmit axial and torsional forces from the proximal section 170 located outside of the patient's body to the distal section 172 of the elongated member 168, which is inserted into the body during the procedure. A handle 174 disposed on the proximal section 170 of elongated member 168 can be used to manipulate the embolectomy device 164 through the vasculature. Although the elongated member 168 shown in FIG. 14 terminates at the filter basket 166, other embodiments have been envisioned wherein the elongated member 168 extends further in the distal direction. Moreover, in certain embodiments, the elongated member 168 can include one or more radiopaque features to aid in visualizing the device within the body.

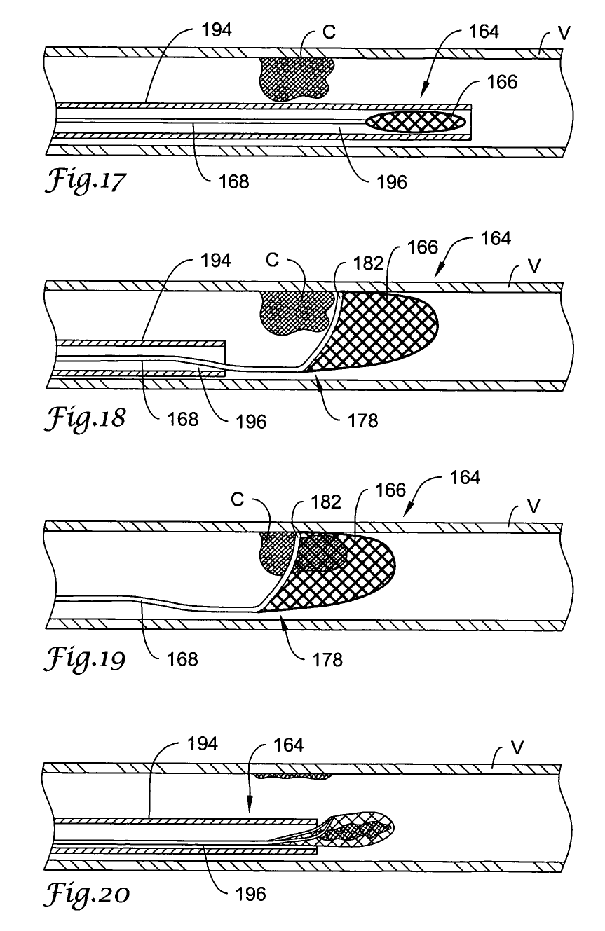

In the exemplary embodiment of FIG. 14, filter basket 166 includes several interconnected filter struts 176 that vary in thickness from the proximal section 178 of the filter basket 166 towards the distal section 180 of the filter basket 166. As shown in FIG. 14, embolectomy device 164 may include a proximal hoop 182 forming a mouth of the filter basket 166 that receives the foreign object as it is dislodged from the vessel wall. The proximal hoop 182 can be configured to self-deploy to an expanded position when deployed from a delivery device (e.g. a microcatheter or guide catheter) after placement within the blood vessel. The proximal hoop 182 can be configured to radially collapse and close the mouth of the filter basket 166 when loaded into the delivery device. As is discussed further with respect to FIGS. 17-20, the proximal hoop 182 may be used to scrape the vessel wall to dislodge the foreign object (e.g. a blood clot) during an embolectomy procedure.

The proximal hoop 182 may include a wire 184 coupled to the distal section 172 of elongated member 168. In the embodiment illustrated in FIG. 14, for example, the wire 184 can be attached to the distal section 172 of elongated member 168 via solder joint 186. In alternative embodiments (not shown), the wire 184 and elongated member 168 can be formed from a single piece of material, or may be formed as an extension of the filter struts 176 used to form the filter basket 166. The proximal hoop 182 can be formed from a resilient material, allowing the proximal hoop 182 and filter basket 166 to be radially collapsed within the delivery device.

Examples of suitable materials used to form the proximal hoop 182 include metals such as nickel-titanium alloy (Nitinol), Beta III Titanium and stainless steel, or polymeric materials such as polyvinyl chloride (PVC). The proximal hoop 182 can also be formed from metal/metal or metal/polymer composites, and can include an anti-thrombogenic layer or coating such as heparin (or its derivatives), urokinase or PPack (dextrophenylalanine proline arginine chloromethylketone) to reduce insertion site thrombosis from occurring. Moreover, the embolectomy device 164 can include a hydrophobic or hydrophilic coating to reduce friction of the device through the vasculature. One or more articulation regions 188 on the proximal hoop 182 may be employed to facilitate the collapse of the filter basket 166 as it is loaded into the delivery device.

FIGS. 15-16 are detailed views, respectively, of a portion of the proximal and distal sections 178,180 of the filter basket 166. As illustrated therein, each section 178,180 can include a plurality of filter struts 176 that are interconnected at several junctures 190 to form a cage-like structure configured to collect a foreign object therein.

The thickness of the filter struts 176 may vary from the proximal section 178 of filter basket 166 towards the distal section 180 of filter basket 166 to alter the stiffness along the length of the embolectomy device 164. For example, as shown in FIG. 15, selective filter struts 176 forming the proximal section 178 of filter basket 166 may have a relatively large thickness t₁ to provide greater rigidity and stiffness to the proximal section 178 of filter basket 166. In contrast, and as shown in FIG. 16, the thickness t₂ of the strands 176 at the distal section 180 of the filter basket 166 may be reduced in comparison to the thickness t₁ at the proximal section 178 to provide greater flexibility towards the distal portion of the embolectomy device 164. In use, the relatively large dimension of the filter struts 176 forming the proximal section 178 of filter basket 166 may enhance the mechanical strength of the embolectomy device 164 at or near the location where the device 164 engages the wall of the blood vessel. The enhanced flexibility at the distal section 180 of the filter basket 166, in turn, facilitates navigation of the embolectomy device 164 through relatively small or tortuous vessels.

The thickness of the filter struts 176 can be reduced gradually from the proximal section 178 towards the distal section 180 of the filter basket 166, producing a gradual transition in stiffness and rigidity along the length of the embolectomy device 164. For example, the thickness of each filter strut 176 can be reduced along the length of the filter basket 166 such that the proximal end of the filter basket 166 has the greatest stiffness, whereas the distal end of the filter basket 166 has the greatest flexibility. The thickness of the filter struts 176 can also be selectively reduced such that only some of the struts in a particular section (e.g. the distal section 180) are reduced in dimension.

Although the structural properties of the embolectomy device 164 may be controlled via the use of filter struts of varying thickness, it should be understood that other factors could be altered to affect the characteristics of the device. For instance, the number of filter struts forming each section may be selected to impart a particular stiffness characteristic to the filter basket. The geometry and material composition of the filter struts, and the number of junctures interconnecting each strut, may also be selected to alter the mechanical properties of the device. For example, although the particular filter struts 176 illustrated in FIGS. 14-16 have a substantially rectangular transverse cross-sectional shape, other shapes such as circular, oval, triangular, etc. may be employed.

Embolectomy device 164 can further include one or more features to enhance the radiopacity of the device within the body. For example, as shown in FIG. 15, several radiopaque markers 192 placed on selective filter struts 176 forming the proximal section 178 of filter basket 166 can be used in conjunction with a fluoroscopic monitor to visualize the location of the embolectomy device 164 within the body. The radiopaque markers 192 can include a band or layer of a radiopaque material such as gold, platinum, tantalum, tungsten, or other suitable radiographically visual material used in the art. The radiopaque markers 192 can be placed flush within an inlet or recess (not shown) formed on the outer surface of the filter strut 176 such that the radiopaque markers 192 do not substantially increase the thickness of the strut 176.

Although the use of radiopaque markers is specifically illustrated in FIG. 15, other radiopaque features may be employed to radiographically visualize the embolectomy device within the blood vessel. In certain embodiments, for example, the material(s) used to form the filter struts may have radiopaque properties that allow the filter struts to be visualized within the body using a fluoroscope. Radiopaque coatings placed about selective filter struts may also be used to facilitate visualization.

Formation of the filter basket 166 may be accomplished by a laser machining process or other suitable manufacturing method. In one exemplary method of manufacture, a workpiece of metallic tubing having a uniform wall thickness can be cut with the aid of a laser to form the various filter struts and junctures forming the filter basket. In an alternative method, a foil or flat sheet of uniform thickness material can be cut with a laser to form the filter struts and junctures, and then rolled into a tubular shape and joined to form the filter basket. The metallic tubing, foil, or flat sheet can be reduced in width from one end to the opposite end such that, when formed, the filter basket has a tapered shape from the proximal end towards the distal end.

Once cut, selective portions of the filter basket are then masked, and a process such as microblasting, chemical etching, or electropolishing can be used to reduce the wall thickness of the unmasked filter struts. In a microblasting process, for example, selective filter struts may be temporarily masked to preserve their shape, and a dry abrasive powder can be ejected through a nozzle and impinged upon the unmasked struts to reduce their thickness. The amount of thickness reduction can be controlled by varying the volume, pressure and duration the abrasive powder is placed into contact with the unmasked filter struts. Once the filter struts have been reduced to the desired dimension, the temporary masks can be removed. The filter basket can then be attached to the elongated member by using solder, crimping, brazing, adhesive, or other suitable bonding technique. In use, the reduction in dimension at the unmasked areas imparts flexibility to the filter basket, allowing the basket to bend or flex more easily as the embolectomy device is advanced through the vasculature.

Referring now to FIGS. 17-20, an exemplary method of retrieving a foreign object within a blood vessel will now be described with respect to embolectomy device 164 described herein. In a first position illustrated in FIG. 17, embolectomy device 164 may be radially collapsed and loaded into a delivery device 194 having an internal lumen 196, and advanced to a location distal to a blood clot C or other foreign body attached along the wall of the blood vessel V. As shown in FIG. 17, delivery device 194 may be dimensioned to cross the site of the blood clot C without dislodging the blood clot C from the vessel wall. The relatively flexible distal section 180 of the filter basket 166 facilitates insertion of the embolectomy device 10 through tortuous and narrowed vessels.

In a second position illustrated in FIG. 18, delivery device 194 is withdrawn proximally, or alternatively, the embolectomy device 164 is advanced distally, causing the filter basket 166 to deploy from the inner lumen 196 of the delivery device 194 and self-expand in the blood vessel V. With the filter basket 166 in a deployed position distal the blood clot C, the operator next retracts the elongated member 168 proximally to disengage the blood clot C from the vessel wall. As the embolectomy device 164 is retracted, the blood clot C initially contacts the proximal hoop 182 at the proximal section 178 of the filter basket 166. Continued retraction of the embolectomy device 164 in the proximal direction causes the blood clot C to become severed from the vessel wall and become entrained within the filter basket 166, as shown in FIG. 19. The relatively large dimension of the filter struts 176 at the proximal section 178 of the filter basket 166 prevents the embolectomy device 164 from deflecting away from the vessel wall as it engages the blood clot C. At the conclusion of the procedure, the embolectomy device 164 and entrained blood clot C can be retracted up to the distal end of the delivery device 194, as shown in FIG. 20, and subsequently removed from the body.

Although the exemplary method illustrated in FIGS. 17-20 shows the advancement of the delivery device 194 beyond the site of the blood clot C prior to deployment, other methods of delivering the embolectomy device 164 to the site of the blood clot are contemplated. In one method, for example, the delivery device 194 and collapsed embolectomy device 164 can be advanced within the blood vessel to a location proximal the blood clot C. Holding the elongated member 168 stationary, the delivery device 194 can be withdrawn in the proximal direction, causing the embolectomy device 164 to eject from the internal lumen 196 and deploy in the blood vessel V. Once deployed, the embolectomy device 164 can be advanced across the site of the blood clot until the proximal hoop 182 is disposed distally of the blood clot in a position similar to that depicted in FIG. 18. The embolectomy device 164 can then be urged proximally to dislodge and capture the blood clot.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. Changes may be made in details, particular in matters of size, shape, and arrangement of parts without exceeding the scope of the invention. It will be understood that this disclosure is, in many respects, only illustrative.

What is claimed is:

1. A medical device for removing a foreign object from a body lumen, comprising:
an elongated member having a proximal section and a distal section;
a support frame attached to the distal section of the elongated member; and
a filter basket having a plurality of filter struts for capturing the foreign object, said plurality of filter struts including a proximal set of filter struts configured to attach the filter basket to a portion of the support frame, and a distal set of filter struts configured to couple the filter basket to the distal section of the elongated member.

2. The medical device of claim 1, wherein the filter basket and support frame are configured to self-expand from a collapsed position to an expanded position when deployed in the body lumen.

3. The medical device of claim 1, wherein said elongated member is a pusher wire.

4. The medical device of claim 1, wherein said support frame includes a proximal hoop and at least one rail member.

5. The medical device of claim 4, wherein said at least one rail member includes a left rail member and a right rail member.

6. The medical device of claim 5, wherein said left and right rail members are arcuately shaped.

7. The medical device of claim 4, wherein the distal set of filter struts and at least one rail member are attached to a bushing slidably disposed about the elongated member.

8. The medical device of claim 1, wherein the support frame includes a superelastic alloy.

9. The medical device of claim 1, wherein the proximal section of the filter basket has a generally open configuration.

10. The medical device of claim 1, wherein the distal section of the filter basket has a generally closed configuration.

11. The medical device of claim 1, wherein the filter basket includes a superelastic alloy.

12. The medical device of claim 1, wherein said proximal set of struts includes four proximal struts.

13. The medical device of claim 1, wherein said proximal set of struts includes two proximal struts.

14. The medical device of claim 1, wherein the support frame and filter basket are each formed of a flat sheet or tubular member.

15. The medical device of claim 14, wherein the support frame and filter basket are formed by a laser cutting or etching process.

16. The medical device of claim 1, wherein the filter basket is formed from a single workpiece.

17. The medical device of claim 1, wherein the support frame and filter basket are each formed of wire or ribbon.

18. The medical device of claim 17, wherein the wire or ribbon forming the filter basket has a smaller transverse cross-sectional area than the wire or ribbon forming the support frame.

19. The medical device of claim 1, wherein at least one of said plurality of filter struts has an undulating shape.

20. The medical device of claim 1, further including a polymeric web covering coupled to the filter basket.

21. A medical device for removing a foreign object from a body lumen, comprising:
an elongated member having a proximal section and a distal section;
a support frame attached to the distal section of the elongated member, said support frame including a proximal hoop and a plurality of rail members; and
a filter basket operatively coupled to the support frame and having a plurality of filter struts for capturing the foreign object, said plurality of filter struts including a proximal set of filter struts configured to attach a proximal section of the filter basket to said at least one rail member, and a distal set of filter struts configured to attach a distal section of the filter basket to a bushing slidably disposed about the distal section of the elongated member.

22. The medical device of claim 21, wherein the filter basket and support frame are configured to self-expand from a collapsed position to an expanded position when deployed in the body lumen.

23. The medical device of claim 21, wherein said elongated member is a pusher wire.

24. The medical device of claim 21, wherein each of said plurality of rail members is arcuately shaped.

25. The medical device of claim 21, wherein the support frame includes a superelastic alloy.

26. The medical device of claim 21, wherein the proximal section of the filter basket has a generally open configuration.

27. The medical device of claim 21, wherein the distal section of the filter basket has a generally closed configuration.

28. The medical device of claim 21, wherein the filter basket includes a superelastic alloy.

29. The medical device of claim 21, wherein said proximal set of struts includes four proximal struts.

30. The medical device of claim 21, wherein said proximal set of struts includes two proximal struts.

31. The medical device of claim 21, wherein the support frame and filter basket are each formed of a flat sheet or tubular member.

32. The medical device of claim 31, wherein the support frame and filter basket are formed by a laser cutting or etching process.

33. The medical device of claim 21, wherein the filter basket is formed from a single workpiece.

34. The medical device of claim 21, wherein the support frame and filter basket are each formed of wire or ribbon.

35. The medical device of claim 34, wherein the wire or ribbon forming the filter basket has a smaller transverse cross-sectional area than the wire or ribbon forming the support frame.

36. The medical device of claim 21, wherein at least one of said plurality of filter struts has an undulating shape.

37. The medical device of claim 21, further including a polymeric web covering coupled to the filter basket.

38. A medical device for removing a foreign object from a body lumen, comprising:
an elongated member having a proximal section and a distal section; and
a filter basket operatively coupled to the distal section of the elongated member, said filter basket including a plurality of interconnected filter struts forming a number of basket cells for capturing the foreign object.

39. The medical device of claim 38, wherein the filter basket is configured to self-expand from a collapsed position to an expanded position when deployed in the body lumen.

40. The medical device of claim 38, wherein said elongated member is a pusher wire.

41. The medical device of claim 38, wherein the filter basket includes a proximal section, a distal section, and an inner lumen.

42. The medical device of claim 41, wherein the proximal section of the filter basket has a generally open configuration.

43. The medical device of claim 41, wherein the distal section of the filter basket has a generally closed configuration.

44. The medical device of claim 41, wherein selective filter struts forming the distal section of the filter basket are reduced in thickness.

45. The medical device of claim 38, wherein the basket cells are configured to displace in multiple directions.

46. The medical device of claim 38, wherein said plurality of interconnected filter struts are formed from a single workpiece.

47. The medical device of claim 38, wherein the filter basket includes a superelastic alloy.

48. The medical device of claim 38, further including a polymeric web covering coupled to the filter basket.

49. A medical device for removing a foreign object from a body lumen, comprising:
an elongated member having a proximal section and a distal section; and
a filter basket operatively coupled to the distal section of the elongated member, said filter basket including a plurality of interconnected filter struts forming a proximal section and a distal section, wherein selective filter struts forming the distal section of said filter basket are reduced in dimension.

50. The medical device of claim 49, further including a proximal hoop coupled to the filter basket.

51. The medical device of claim 49, further including a radiopaque marker on at least one of said plurality of interconnected filter struts.

52. A method of forming an embolectomy device having variable wall thickness, comprising the steps of:
providing a workpiece of uniform thickness;
machining the workpiece to form a filter basket having a plurality of filter struts; and
selectively reducing the thickness of at least one of said plurality of filter struts to impart a desired characteristic to the embolectomy device.

53. The method of claim 52, wherein said machining step is performed by a laser machining process.

54. The method of claim 52, wherein said machining step is performed by an etching process.

55. The method of claim 52, wherein said reducing step comprises the steps of:
masking the filter struts forming the proximal section of the filter basket; and
removing a portion of the unmasked filter struts on the distal section of the filter basket.

56. The method of claim 55, wherein the step of removing a portion of the unmasked filter struts includes microblasting the filter struts.

57. The method of claim 55, wherein the step of removing a portion of the unmasked filter struts includes electropolishing the filter struts.
